(19) United States
(12) Patent Application Publication
(10) Pub. No.: US 2011/0046346 A1
(43) Pub. Date: Feb. 24, 2011

(54) BIOMATERIAL WITH FUNCTIONALISED SURFACES

(75) Inventors: Andrew William Lloyd, Alfriston (GB); George William John Olivier, Haywards Heath (GB); Guy Standen, Steyning (GB); Matteo Santin, Brighton (GB); Steven Thomas Meikle, Brighton (GB)

Correspondence Address: VOLPE AND KOENIG, P.C., UNITED PLAZA, 30 SOUTH 17TH STREET, PHILADELPHIA, PA, 19103, US

(73) Assignee: UNIVERSITY OF BRIGHTON, Brighton (GB)

(21) Appl. No.: 12/862,233

(22) Filed: Aug. 24, 2010

Related U.S. Application Data
(63) Continuation of application No. 12/517,705, filed on Oct. 12, 2009.

(30) Foreign Application Priority Data
Dec. 6, 2006 (GB) 0624423.0

(51) Int. Cl. C07K 14/00; C07C 237/10; C07H 99/00; B82Y 30/00

(52) U.S. Cl. 530/324; 564/153; 536/1.11; 977/754

(57) ABSTRACT

There is provided a biomaterial having a functionalised surface which comprises bi-functional semi-dendrimers. The biomaterial may be ceramic, metallic and/or polymeric. It will usually be in the form of a solid, but could be a semi-solid or hydrogel. There is also provided a method of making a biomaterial having a functionalised surface which comprises bi-functional semi-dendrimers, said method comprising adsorbing, grafting or synthesising in situ bi-functional semi-dendrimers onto the surface of a biomaterial. There is further provided a biomedical device which is coated with or formed from a biomaterial having a functionalised surface which comprises bi-functional semi-dendrimers. The biomedical device may be a medical implant, for example, such as a stent, artificial hip joint or replacement heart valve. FIG. 1 is a schematic representation of a bi-functional semi-dendrimer structure suitable for biomaterial functionalisation according to the present invention. B represents a group with functionality bridging the dendrimer to the biomaterial; D represents a group with functionality driving the biorecognition of the biomaterial or other bioactive processes in which it is involved. Examples of D groups include peptides, amino acids, carbohydrates, antibiotics, etc.

This application is a continuation application of U.S. application Ser. No. 12/517,705, which was filed Oct. 12, 2009 as a 35 U.S.C. 371 national phase application of PCT/GB2007/050741, which was filed Dec. 5, 2007 both of which are incorporated herein by reference as if fully set forth

This invention relates to the functionalisation of biomaterials, in particular the surfaces of biomedical devices made from biomaterials, such as implants, through the use of bi-functional semi-dendrimers.

Biomaterials are polymeric, metallic and/or ceramic materials destined to contact body tissues in biomedical applications. They are used for the manufacture of medical devices which are implanted in the human or animal body to replace damaged tissues. In many clinical applications, the successful implantation of a medical device depends on its integration with the surrounding tissues. The control of interactions between the biomaterial solid surfaces of an implant and the chemical, biochemical and cellular components of the biological environment, which surround the implant, is a fundamental step of this integration process. Indeed, biomedical implants can integrate with the surrounding tissue only by allowing the adhesion, proliferation and differentiation of the tissue cells responsible for the regeneration of the tissue at the implant/tissue interface [1]. Furthermore, in the case of implants for bony tissue, integration is also achieved by binding of the mineralised extracellular matrix to the implant surface [2].

Methods have been developed to functionalise the surface of biomedical implants with molecules able to bind specific proteins [3, 4] to encourage the adhesion of cells or the deposition of mineral phase [5, 6, 7]. For example, the mineralization of biomaterial surfaces has been pursued by etching methods [6] or by coating of the surface with calcium-binding phospholipids such as phosphatidylserine [7]. These methods rely on the adsorption [7] or grafting [3, 4, 5] of relevant biomolecules on the biomaterial surface through linear spacers or by chemical treatment of the surface [6]. More recently, synthetic molecules such as agmatine have been used which can mimic the amino acid sequences recognised by specific cell receptors such as the RGD domain [8].

Other biomaterial surface treatments have been developed to create a nanotopography which can mimic that of the tissue extracellular matrix [9, 10]. However, most of these treatments produce surface modifications of the biomaterials which lack the ordered three-dimensional (3D) molecular nanoarchitecture and/or the molecular flexibility typical of the naturally occurring tissue extracellular matrix.

Indeed, it is widely recognised that the specificity of the biorecognition process can be enhanced if the underlying chemical and biochemical interactions are accompanied by an appropriate nanostructure, which improves the exposure of the functionalities to the surrounding environment and/or mimics the architectures of biological structures which have naturally evolved to facilitate specific bio-interactions [1].

Dendrimers and semi-dendrimbers are highly and 3-D ordered, hyperbranched polymers forming nanostructures with controllable physico-chemical properties [11, 12]. They can be obtained from monomeric molecules of different types sharing the ability of developing into branching macromolecules. Dendrimers have been obtained from synthetic molecules (e.g. polyamido amine, PAMAM) as well as from amino acids (e.g. polylysine) and carbohydrates [11, 12, 13]. There are two main methods to synthesise dendrimers [11]:
(i) The divergent synthesis where a core molecule with multiple reactive sites is used to form chemical bonding with a reactant, and where the formed complex is later reacted with a molecule capable of generating another branching point.
(ii) The convergent synthesis where fragments of dendrimers are added to the core molecules and thus assembled.

When the synthesis is performed in the liquid phase, although the shape and symmetry of the dendrimer depends on the physico-chemical properties of the molecules used for its synthesis, the polymer branching generally leads to an open ball, spherical structure [11]. Conversely, when the synthesis is performed in the solid phase the branching polymer develops a dome-like (semi-sphere) or tree-like structure, the semi-dendrimer [12].

By both methods (i) and (ii) it is possible to obtain dendrimers (or semi-dendrimers) with several branching levels (referred to as generations, Gₙ). The synthesis of dendrimers up to nine generations (G₉) has indeed been reported.

From a biotechnological perspective both dendrimers and semi-dendrimers offer a unique opportunity to expose functionalities suited to favour bio-interactions and a nanostructure to control distance and steric specificity [14].

Dendrimers have been mainly proposed as carriers for the delivery of nucleic acids and drugs [15]. In particular, PAMAM dendrimers can bind DNA because of their overall positive charge which establishes ionic interactions with the negative charge of nucleic acids [15]. However, it has been shown that dendrimer nanoarchitecture also contributes to their DNA-binding potential [16]. The ability of PAMAM dendrimers to bind DNA has been exploited to capture DNA and other nucleic acids. For these applications, microchannel surfaces have been functionalised with dendrimers for that purpose [17]. Semi-dendrimers have been investigated as a possible way to increase the affinity of specific bioligands to cell receptors by functionalising the last branching generation of the dendrimer with the targeted bioligand [14].

The binding of dendrimers to solid surfaces is usually obtained by prior functionalisation of the surface with a silanisation reaction which grafts a linear molecule exposing an amino group at its end [3, 4, 17]. Later, the amino group is bridged to the dendrimer by glutaraldehyde; the aldehyde group of glutaraldehyde reacts with the amino groups of both the silanising molecule and dendrimers such as the PAMAMs [17].

According to the present invention there is provided a biomaterial having a functionalised surface which comprises bi-functional semi-dendrimers. The biomaterial may be ceramic, metallic and/or polymeric. It will usually be in the form of a solid, but could be a semi-solid or hydrogel.

According to another embodiment of the present invention there is provided a method of making a biomaterial having a functionalised surface which comprises bi-functional semi-dendrimers, said method comprising adsorbing, grafting or synthesising in situ bi-functional semi-dendrimers onto the surface of a biomaterial.

According to still another embodiment of the present invention there is provided a biomedical device which is coated with or formed from a biomaterial having a functionalised surface which comprises bi-functional semi-dendrimers. The biomedical device may be a medical implant, for example, such as a stent, artificial hip joint or replacement heart valve.

The biomaterials of the present invention are capable of specific bio-interactions with chemical, biochemical and cellular components of the human and animal biological systems relevant to implants and tissue engineering constructs. The functionalised surface of the biomaterial and/or of the biomedical device coated with or formed from the biomaterial may be a 3D nano-structured surface which mimics that of the tissue extracellular matrix. A bi- (or dual) functionality in the semi-dendrimer structure is created by a core molecule exposing a chemical or biochemical group different from that exposed on the last branching generation of the semi-dendrimer. In general, the chemical or biochemical group exposed by the core molecule at the root of the molecular tree (the first functionality) will facilitate the grafting of the semi-dendrimer to the surface of the biomaterial, while the functionality exposed on the last branching generation (the second functionality of the bi-functional semi-dendrimer) will regulate its bio-interactions.

The present invention will now be described in more detail by reference to the following Examples and the accompanying Figures: —

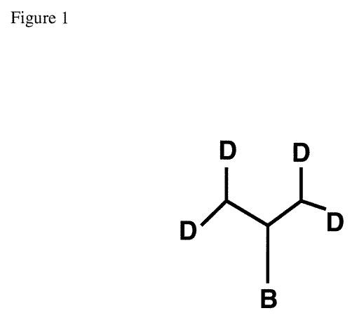

FIG. 1: A schematic representation of a bi-functional semi-dendrimer structure suitable for biomaterial functionalisation according to the present invention. B represents a group with functionality bridging the dendrimer to the biomaterial; D represents a group with functionality driving the biorecognition of the biomaterial or other bioactive processes in which it is involved. Examples of D groups include peptides, amino acids, carbohydrates, antibiotics, etc.

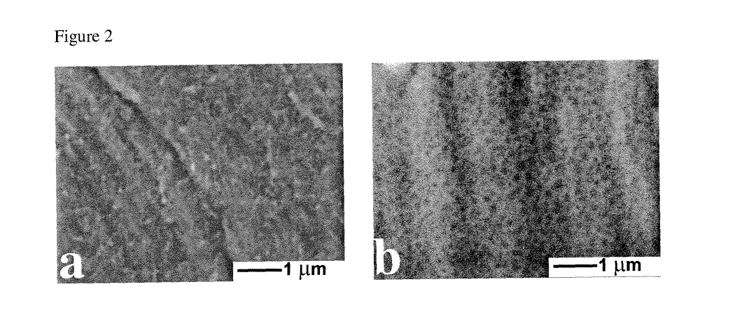

FIG. 2: Images produced by scanning electron microscopy of a biomaterial surface; wherein (a) is a non-functionalised surface and (b) is a semi-dendrimer functionalised surface according to the present invention.

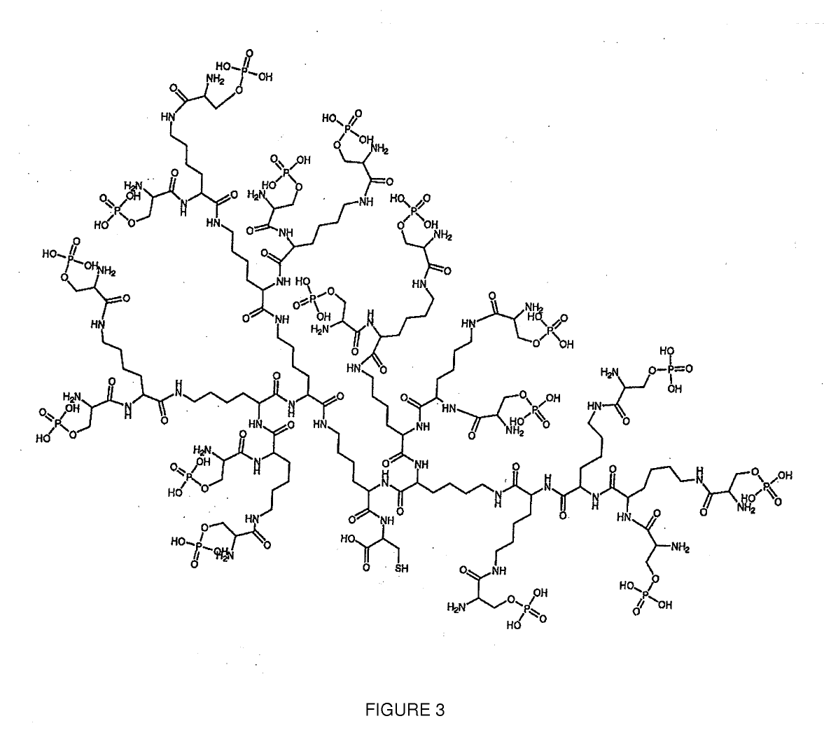

FIG. 3: The molecular structure of a bi-functional G₃ semi-dendrimer exposing a phosphoserine group.

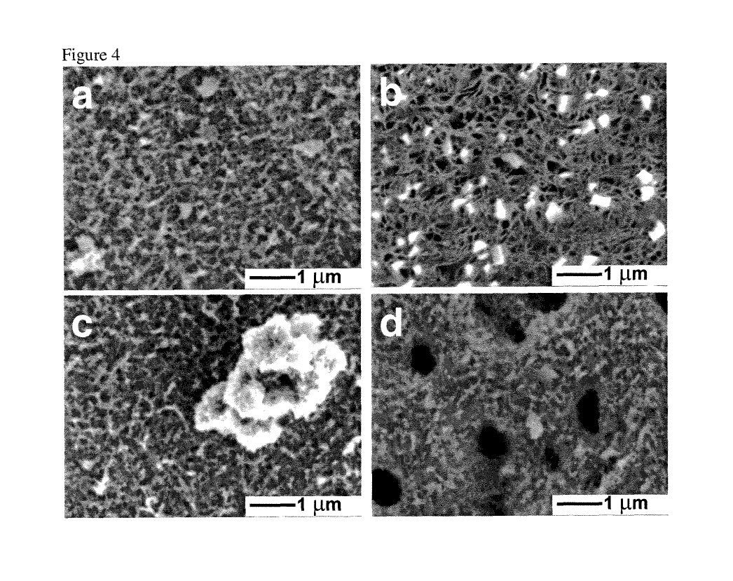

FIG. 4: Scanning electron micrographs of the different stages of mineralization of a biomaterial surface after its functionalisation with bi-functional G₃ semi-dendrimers exposing a phosphoserine group; wherein (a) is the mineralising nanostructured semi-dendrimer network after 48 h incubation in simulated body fluid, (b) shows the formation of a discrete calcium phosphate crystal on the semi-dendrimer network after 48 h incubation in simulated body fluid, (c) is the crystal seed formed on the coating surface and (d) shows organised mineralised 3D nanostructure.

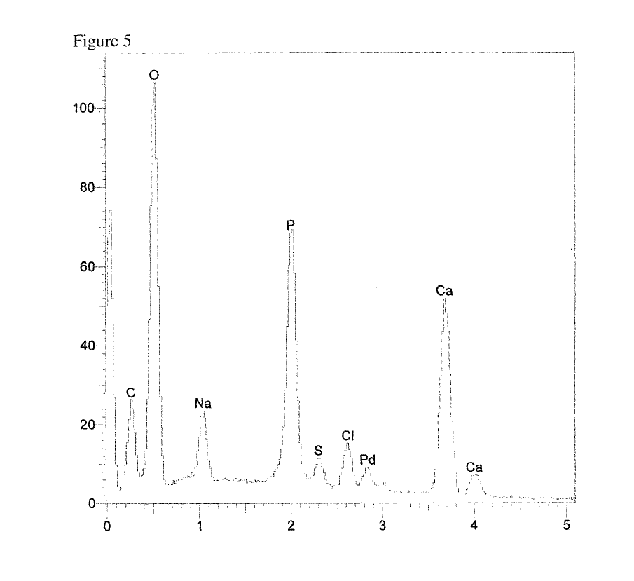

FIG. 5: A typical Energy-Dispersive X-ray (EDX) analysis of the mineralised phosphoserine semi-dendrimer coating of FIG. 4, showing a presence of calcium and phosphorus, after its exposure to simulated body fluid.

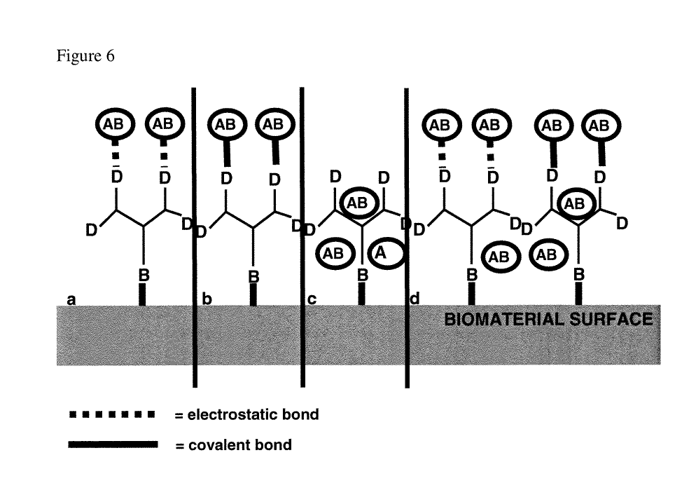

FIG. 6: A schematic representation of a biomaterial surface functionalised with bi-functional semi-dendrimers exposing antibacterial agents by (a) non-specific (e.g. electrostatic) interactions, (b) covalent binding, (c) entrapment and (d) a combination of them, according to the present invention.

### EXAMPLE

### Bi-Functional Semi-Dendrimers

Methods. Polylysine and PAMAM semi-dendrimers are synthesised using commercially-available solid-phase matrices. In the case of PAMAM semi-dendrimers, the synthesis is based on the conventional dendrimer synthesis divergent method where a Michael's addition reaction is followed by the elongation of the molecular branch with a diamide addition. Different amino acids are used as core molecules to obtain semi-dendrimers exposing suitable functional groups at their root, such as —NH₂, —SH and —OH. Such functional groups become exposed after the semi-dendrimer is cleaved from the solid phase synthesis matrix and are made available for grafting onto the biomaterial surface. The second functionality is obtained by adding amino acid or other molecules able to support a specific bio-interaction. Typical examples of biomolecules exposed at the last branching generation of the semi-dendrimers are reported in Table 1 and include, for example, the addition of a phosphoserine group able to bind calcium (see Example 3).

TABLE 1
Typical biofunctionalities exposed on semi-dendrimers.
Footnotes refer to examples of use of biospecific molecules
in biomaterial field.

| Functional group | Type | Function |
|---|---|---|
| Phosphoserine^{a} | Amino acid | Mineralization |
| RGD^{b} | Peptide | Cell recognition |
| FHRRIKA | Peptide | Osteoblast migration |
| KRSR | Peptide | Osteoblast recognition |
| Spermidine | Polyamine | Substrate for clotting enzyme |
| Agmatine^{c} | Synthetic monomer | Cell recognition |
| | mimicking RGD | |
| Galactose | Sugar | Cell recognition |
| Glucosamine | Sugar | Cell recognition |
| Glutathione | Tripeptide | Antioxidant |

^{a}See reference 7
^{b}See references 3, 4, 6
^{c}See reference 8

A typical protocol of synthesis for a bi-functional PAMAM semi-dendrimer includes the following steps:

### Attachment of the Rink-Amide-Linker and the Core Molecule (Fmoc-Gly) or Peptide (Fmoc-Ending Peptide)

Rink-Amide-Linker (0.2 g, 0.4 mmol), TCTU (0.14 g, 0.4 mmol) and DIPEA (0.11 ml, 0.4 mmol) in DMF (15 ml) for 12 h at room temperature, then wash with DMF.
20% piperidine in DMF for 2×15 min, then wash with DMF.
Fmoc-Gly-OH or Fmoc-peptide-OH (0.1 g, 0.4 mmol), TCTU (0.14 g, 0.4 mmol) in DMF (10 ml) for 2 h at room temperature, then wash with DMF.
20% piperidine in DMF for 2×15 min, then wash with DMF and methanol.

### Dendrimer Synthesis

Methyl acrylate (10 ml) and MeOH (20 ml) (ratio=1:2), 24 h at 60° C., wash with methanol.
1,3-Diaminopropane (20 ml) (DAP) and MeOH (20 ml) (ratio=1:1), 24 h at 60° C., wash with methanol.

### Dendrimer Biofunctionalisation

Relevant functional molecules (e.g. amino acids, peptides, carbohydrates, etc.) are covalently bound to the semi-dendrimer's uppermost generation by conventional and appropriate chemical methods [13, 14].

In the case of the bi-functional polylysine semi-dendrimers, the method consists of a conventional solid-phase polypeptide synthesis where, by a sequence of amino acid protection/deprotection steps, polylysine molecules are added to form branched polymeric structures of up to five branching generations. The synthesis was performed by the following protocol:

### Synthesis of a Polylysine Semi-Dendrimer Using an Automated Peptide Synthesiser

Peptide synthesis resin (0.5 g, 0.1 mmol (—NH₂)) was swollen with DMF on the peptide synthesiser.

Vials were then loaded onto the peptide synthesiser as follows: —
One vial containing Rink Amide linker (215.8 mg, 0.4 mmol) and TCTU (142.2 mg, 0.4 mmol).
One vial containing Fmoc-Cys(Trt)-OH (234.3 mg, 0.4 mmol) and TCTU (142.2 mg, 0.4 mmol).
Fmoc-Lys(Fmoc)-OH (236.3 mg, 0.4 mmol) and TCTU (142.2 mg, 0.4 mmol) dependent on the generation of polylysine required (e.g. 11 vials are required for G₃ polylysine).
Then each vial in turn followed the sequence: —
   (i) Dissolution of contents with DMF.
   (ii) Reaction with the peptide synthesis resin.
   (iii) Wash resin with DMF.
   (iv) Wash resin with 20% piperidine in DMF to deprotect amino acid.
   (v) Repeat steps (i)-(iv) for next vial.
   (vi) Addition of the final amino acid does not involve step (iv).
      Cleavage of Peptide from Peptide Synthesis Resin
Transfer the resin to a fitted syringe.
Wash the resin with 5×8 cm³ dichloromethane, methanol and finally diethyl ether and dry under a nitrogen stream.
The washed resin was then deprotected using a standard deprotection method dependent on the protecting groups used throughout the synthesis and purified prior to analysis by mass spectrometry.

Results. FIG. 1 shows the schematic structure of a typical bi-functional semi-dendrimer used in the present invention. Table 2 shows the mass spectrometry data of a typical polylysine G₃ bi-functional semi-dendrimer obtained from a cysteine core molecule exposing a thiol group at its molecular root. In the case of phosphoserine terminal functionalisation the mass of the final semi-dendrimer was 4683.1.

TABLE 2
G₃ polylysine semi-dendrimer mass spectrometry data

| K's | M | M + H | M + 2H | M + 3H | M + 4H | M + 5H |
|---|---|---|---|---|---|---|
| 1 | 249.1317 | 250.1397 | 125.5739 | 84.0519 | 63.29093 | 50.83434 |
| 2 | 377.2267 | 378.2347 | 189.6214 | 126.7502 | 95.31468 | 76.45334 |
| 3 | 505.3217 | 506.3297 | 253.6689 | 169.4486 | 127.3384 | 102.0723 |
| 4 | 633.4167 | 634.4247 | 317.7164 | 212.1469 | 159.3622 | 127.6913 |
| 5 | 761.5117 | 762.5197 | 381.7639 | 254.8452 | 191.3859 | 153.3103 |
| 6 | 889.6067 | 890.6147 | 445.8114 | 297.5436 | 223.4097 | 178.9293 |
| 7 | 1017.702 | 1018.71 | 509.8589 | 340.2419 | 255.4334 | 204.5483 |
| 8 | 1145.797 | 1146.805 | 573.9064 | 382.9402 | 287.4572 | 230.1673 |
| 9 | 1273.892 | 1274.9 | 637.9539 | 425.6386 | 319.4809 | 255.7863 |
| 10 | 1401.987 | 1402.995 | 702.0014 | 468.3369 | 351.5047 | 281.4053 |
| 11 | 1530.082 | 1531.09 | 766.0489 | 511.0352 | 383.5284 | 307.0243 |
| 12 | 1658.177 | 1659.185 | 830.0964 | 553.7336 | 415.5522 | 332.6433 |
| 13 | 1786.272 | 1787.28 | 894.1439 | 596.4319 | 447.5759 | 358.2623 |
| 14 | 1914.367 | 1915.375 | 958.1914 | 639.1302 | 479.5997 | 383.8813 |
| 15 | 2042.462 | 2043.47 | 1022.239 | 681.8286 | 511.6234 | 409.5003 |

### EXAMPLE 2

### Surface Functionalisation by Bi-Functional Semi-Dendrimers

Method. Bi-functional semi-dendrimers of Example 1 are in-situ synthesised onto the biomaterial surface as described in Example 1. Prior to in-situ synthesis the biomaterial surface can be activated by conventional chemical methods to obtain functional groups, such as —OH, —NH₂ or —SH groups, which are required for the grafting of the core molecule or peptide. Activation methods include, for example, silanisation reactions the use of dialdehyde and surface etching (such as, alkali etching and plasma etching).

Typical examples of biomaterial surface activation as reported in the literature [3, 4, 6] are:

Silanisation Reaction
0.1% 3-Aminopropyltrimethoxysilane (APTMS) or 3-aminopropyltriethoxysilane (APTES) solution in toluene (100 ml) refluxing for 2 h at 110° C., wash with toluene or methanol.
Silanisation may also be performed in the gaseous state using 3-aminopropyltrimethoxysilane (APTMS) or 3-aminopropyltriethoxysilane (APTES) applied under a vacuum.

### Dialdehyde Activation

Dialdehyde surface activation was obtained by incubation of the clean surfaces with dialdehyde such as glutaraldehyde or genipin solution at different concentrations [e.g. 0.1%, 0.5%, and 2.5% (v/v)] in distilled water for 20-30 minutes. Alternatively, the biomaterial surface is exposed to an environment of saturated dialdehyde for different times at room temperature.
The activated surface was washed thoroughly with distilled water.

### Alkali Etching

The biomaterial surface is treated with alkali (NaOH, KOH) at different concentrations in the range 0.1 to 5 M, 1 h, room temperature.

After activation the biomaterial surface is equilibrated with methanol for 30 min at room temperature. The in-situ solid phase synthesis of the bi-functional semi-dendrimer is then performed as reported in Example 1.

Alternatively, bi-functional semi-dendrimers are grafted onto solid surfaces of biomaterials by different chemical reactions including the use of (i) the aldehyde group of a dialdehyde (e.g. glutaraldehyde and genipin) to the semi-dendrimer —OH or —NH₂, (ii) the reaction of —SH groups exposed on the solid surface as well as on the semi-dendrimer core structure. Metal oxides and gold surfaces, as well as polymeric materials, can be functionalised by these methods. A typical example of a grafting protocol includes the following steps:
Activated biomaterial surfaces were washed thoroughly with warm (40° C.) distilled water, then with distilled water at a stabilized temperature (25° C.).
The surface was equilibrated with buffers such as 10 mM Tris-HCl, 20 mM MgSO₄ (pH=7.0-7.5) [Tris-Mg].
Semi-dendrimer solution (30 μl+120 μl Tris buffer) was then introduced in the reaction vessel. To saturate the functionalised surface with semi-dendrimers, the reaction was performed by intercalating 10-15 min incubation steps with aliquots of semi-dendrimer solutions with washing of non-bound material by buffer.

In a third method, biomaterial functionalisation can be achieved by physical adsorption of the semi-dendrimers of Example 1 on the exposed surface. This is achieved by incubating the biomaterial surface in a semi-dendrimer solution for different times at room temperature. Different incubation times and semi-dendrimer solution concentrations will lead to coatings of different thickness. Electrostatic and/or hydrophobic as well as hydrogen bonding drive this process depending on the physico-chemical characteristics of the exposed surface and adsorbing semi-dendrimers. The formed semi-dendrimer mono- or multi-layer can also be stabilised by its treatment with crosslinking agents, thereby forming a nanostructured network on the surface. Crosslinking agents include, for example, dialdehydes (e.g. glutaraldehyde, formaldehyde, genipin, etc). The crosslinking can be obtained by incubation of the semi-dendrimer-coated biomaterial in a crosslinking agent solution (e.g. 2.5% by volume glutaraldehyde) or in its saturated atmosphere. Crosslinking of semi-dendrimers functionalised with peptide sequences recognised as a substrate by the clotting enzyme Factor XIII can also be obtained by incubation with solutions of this enzyme or by direct contact with blood.

Results. The semi-dendrimers are in-situ synthesised or grafted on the surface of a biomaterial, such as polymeric and metal biomaterials, to enhance bio-specificity. When compared to a non-functionalised surface (FIG. 2 a), a homogeneous nano-structured network of semi-dendrimers is formed by these methods (FIG. 2 b).

### EXAMPLE 3

### Surface Mineralization of Biomaterials Functionalised by Bi-Functional, Phosphoserine-Exposing Semi-Dendrimers

Method. Bi-functional semi-dendrimers are synthesised as described in Example 1 and their top branching generation functionalised by the addition of a phosphoserine amino acid as shown in FIG. 3. The phosphoserine-exposing semi-dendrimers are in-situ synthesised or grafted onto the surface of biomaterials as described in Example 2.

Mineralization experiments were performed by incubating uncoated biomaterial (e.g. titanium oxide) surfaces and phosphoserine exposing semi-dendrimer-coated surfaces in simulated body fluid for 48 and 72 hours, 37° C., static conditions. The simulated body fluid composition included: 71 mM NaCl, 5 mM KCl, 1.64 mM Na₂HPO₄, 2.36 mM CaCl₂ dissolved in 0.05 M TES buffer, pH 7.2.

Results. FIGS. 4 a-d show the progressive formation of ordered calcium phosphate based mineral phase on a solid surface previously functionalised with phosphoserine-based G₃ semi-dendrimers and subsequently incubated in simulated body fluids with a calcium and phosphorus concentration similar to human body fluids. When phosphoserine coatings were applied as multi-layered coating following the physical adsorption method described in Example 2, a highly organised 3D nano-structure was obtained (FIG. 4 d). In all of the cases, EDX showed the presence of a calcium phosphate-rich mineral phase (FIG. 5).

### EXAMPLE 4

### Cell Adhesion on Biomaterials Functionalised by Bi-Functional, Cell Receptor-Binding Semi-Dendrimers

Method. Bi-functional semi-dendrimers are synthesised as described in Example 1 and their top branching generation exposes a bioligand recognised by cell receptors which include, for example, integrin. The semi-dendrimers are in-situ synthesised or grafted on the surface of a biomaterial as described in Example 2.

Results. Cells were able to adhere and spread uniformly on a smooth titanium surface functionalised with bi-functional semi-dendrimers, while they form clusters on non-functionalised smooth metal surfaces.

### EXAMPLE 5

### Surface Functionalisation by Bi-Functional Semi-Dendrimers Exposing Antibacterial Agents

Method. Bi-functional semi-dendrimers are synthesised as described in Example 1 and their top branching generation exposes an antibacterial agent, such as antibiotic molecules. The semi-dendrimers are in-situ synthesised or grafted on the surface of a biomaterial as described in Example 2 to prevent bacterial infections. Antibacterial agents including, for example, antibiotic and silver ions were bound to the surface exposed bi-functional semi-dendrimer either by non-specific interactions (e.g. electrostatic and/or hydrophobic) or by covalent bonding or by entrapment in the semi-dendrimer branching.

Results. FIGS. 6 a-d show the schematic representations of a biomaterial surface functionalised with bi-functional dendrimers exposing antimicrobial molecules bound by different methods and released upon implantation.

### REFERENCES

1. Castner, D. G., Ratner, B. D (2002) Biomedical surface science: foundation to frontiers. Surf. Sci. 500, 28-60.
2. Takeshita F. et al. (1997) Study of bone formation around dense hydroxyapatite implants using light microscopy, image processing and confocal light microscopy. Biomaterials, 18, 317-322.
3. Nanci A., Wuest J. D., Peru L., Brunet P., Sharma V., Zalzal S. and McKee M. D. (1998) Chemical Modification of Titanium Surfaces for Covalent Attachment of Biological Molecules. Journal of Biomedical Materials Research. 40, 324-335.
4. Nanci et al. U.S. Pat. No. 5,824,651 (1998)
5. Rammelt S et al. (2006) Coating of titanium implants with collagen, RGD peptide and chondroitin sulphate. Biomaterials 27, 5561-5571
6. Kokubo et al. U.S. Pat. No. 5,609,633 (1997)
7. Lloyd et al. (2000) Bioimplant coatings PCT/GB00/03290
8. Franchini J et al. (2006). Synthesis, physico-chemical properties, and preliminary biological characterizations of a novel amphoteric agmatine-based poly(amidoamine) with RGD-like repeating units. Biomacromolecules 7, 1215-1222.
9. Parker J A T C et al. (2002). The effect of bone anchoring and micro-grooves on the soft tissue reaction to implant. Biomaterials 23, 3887-3896
10. Sandrini E. et al. (2005). In vitro assessment of the osteointegrative potential of a novel multiphase anodic spark deposition coating for orthopaedic and dental implants. J Biomed Mater Res B. —Appl Biomat. 73B, 392-399.
11. Hobson L. J., Feast W. J. (1999) Poly(amidoamine) hyperbranched systems: synthesis, structure and characterization. Polymer 40, 1279-1297
12. N. J. Wells et al. Solid-phase dendrimer synthesis. Biopolym. 47, 381-396 (1998).
13. Dubber M., Lindhorst, T. K. (2001) Trehalose-Based Octopus Glycosides for the Synthesis of Carbohydrate-Centered PAMAM Dendrimers and Thiourea-Bridged Glycoclusters. Organic Letters. 3, 4019-4022.
14. Monaghan S. (2001) Solid-Phase Synthesis of Peptide-Dendrimer Conjugates for an Investigation of Integrin Binding. ARKIVOC. 46-53.
15. Tang M. X. et al. (1996) In vitro gene delivery by degraded polyamidoamine dendrimers. Bioconjugate Chemistry 7, 703-714.
16. Tang M. X., Szoka, F. C. (1997) The influence of polymer structure on the interactions of cationic polymers with DNA and morphology of the resulting complexes. Gene Therapy. Vol. 4. pp. 823-832.
17. F. Kazuhisa et al. (2005) Dendrimer-based DNA extraction methods and biochips. US Patent US 200513019.

What is claimed is:

1. A biomaterial having a functionalised surface which comprises bi-functional semi-dendrimers that are capable of specific bio-interactions with chemical, biochemical and cellular components of the human and animal biological systems relevant to implants and tissue engineering constructs;
wherein the bi-functional semi-dendrimers each comprise a core molecule exposing a chemical or biochemical group (the first functionality) different from that exposed on the last branching generation of the semi-dendrimer (the second functionality);
the first functionality facilitating the grafting of the semi-dendrimers to the surface of the biomaterial, while the second functionality regulates their specific bio-interactions.

2. A biomaterial as claimed in claim 1, wherein the core molecule of the semi-dendrimers is formed from PAMAM, polylysine or carbohydrates.

3. A biomaterial as claimed in claim 1, wherein the second functionality is formed from an amino acid, a peptide or polypeptide, a polyamine, a synthetic monomer mimicking RGD or a carbohydrate.

4. A biomaterial as claimed in claim 1, wherein the second functionality is formed from phosphoserine.

5. A biomaterial as claimed in claim 1, wherein the biomaterial is ceramic, metallic or polymeric.

6. A biomaterial as claimed in claim 1, wherein the semi-dendrimer functionalised surface forms a porous nano-structure.

7. A biomaterial as claimed in claim 1, wherein the biomaterial is in the form of a biomedical device.

8. A biomaterial as claimed in claim 7, wherein the biomedical device is an implant or a tissue engineering construct.

9. A biomaterial as claimed in claim 1, wherein the biomaterial is intended for use in cell culture techniques.

10. A method of making a biomaterial as claimed in claim 1, said method comprising adsorbing, grafting or synthesising in situ bi-functional semi-dendrimers onto its surface.
